(19) United States
(12) Patent Application Publication
(10) Pub. No.: US 2025/0241726 A1
(43) Pub. Date: Jul. 31, 2025

(54) COUCH OF MEDICAL IMAGE CAPTURING APPARATUS

(71) Applicant: FUJIFILM Corporation, Tokyo (JP)

(72) Inventor: Natsuki ISHIJIO, Tokyo (JP)

(73) Assignee: FUJIFILM Corporation, Tokyo (JP)

(21) Appl. No.: 19/033,469

(22) Filed: Jan. 21, 2025

(30) Foreign Application Priority Data
Jan. 25, 2024 (JP) 2024-009819

(51) Int. Cl. A61B 90/00; A61B 6/00; A61B 6/04

(52) U.S. Cl. CPC A61B 90/08; A61B 6/0407; A61B 6/4435

(57) ABSTRACT

A couch includes a top plate on which a subject lies, a caster portion that is installed on a floor surface side, an elevating mechanism that is provided between the top plate and the caster portion and is used to adjust a height of the top plate, and a cover that is tubular to cover a periphery of the elevating mechanism and is capable of expanding and contracting vertically according to driving of the elevating mechanism. The cover has an opening and closing portion that is openable and closable and is configured by a cutout longitudinally extending from an upper end on a top plate side to a lower end on a caster portion side.

### CROSS-REFERENCE TO RELATED APPLICATIONS

The present application claims priority under 35 U.S.C. § 119 to Japanese Patent Application No., 2024-009819 filed on Jan. 25, 2024. The above application is hereby expressly incorporated by reference, in its entirety, into the present application.

### BACKGROUND

### 1. Technical Field

The technology of the present disclosure relates to a couch of a medical image capturing apparatus.

### 2. Description of the Related Art

In a medical facility, a medical image capturing apparatus such as a magnetic resonance imaging (MRI) apparatus or a computed tomography (CT) apparatus is used. Such a medical image capturing apparatus comprises a couch. The couch comprises a top plate on which a subject (patient) lies, a pedestal part that is installed on a floor surface side, an elevating mechanism that is provided between the top plate and the pedestal part and is used to adjust a height of the top plate, and a tubular cover that covers a periphery of the elevating mechanism.

The cover has a structure that can expand and contract vertically according to driving of the elevating mechanism. WO2013/042589A, JP2014-121584A, and JP2017-018599A disclose a couch of a medical image capturing apparatus comprising a cover having a bellows structure as a structure that can expand and contract vertically.

### SUMMARY

The cover can be attached and detached to be replaced in a case of being stained and/or damaged. In the related art, in a case where the cover is detached from the couch for replacement, since the cover has a tubular shape, a method of folding the cover and sliding the cover under the pedestal part to be removed as if taking off a skirt has been adopted. However, with this method, a lot of complicated work such as jacking up the pedestal part and inserting a block under the pedestal part to ensure a space under the pedestal part for the cover to pass through is required. Similarly, even in a case where the cover is attached to the couch, a lot of complicated work is required.

The couch of the medical image capturing apparatus has the above-described elevating mechanism, a sliding mechanism for the top plate, or a fitting such as a belt for fixing a position of the subject to the top plate, and thus has a relatively heavy weight. Therefore, in the above-described method in the related art, a burden on a worker is very large. Therefore, there has been a demand for a measure for facilitating attachment and detachment of the cover on the couch of the medical image capturing apparatus.

One embodiment according to the technology of the present disclosure provides a couch of a medical image capturing apparatus in which a cover can be easily attached and detached.

A couch of a medical image capturing apparatus according to an aspect of the present disclosure comprises: a top plate on which a subject lies; a pedestal part that is provided on a floor surface side; an elevating mechanism that is provided between the top plate and the pedestal part and is used to adjust a height of the top plate; and a cover that is tubular to cover a periphery of the elevating mechanism, is capable of expanding and contracting vertically according to driving of the elevating mechanism, and has an opening and closing portion that is openable and closable portion and is configured by a cutout longitudinally extending from an upper end on a top plate side to a lower end on a pedestal part side.

It is preferable that the cover has a bellows structure configured by a flexible sheet and a plurality of frame members extending in a direction intersecting the opening and closing portion.

It is preferable that one end and the other end of the sheet facing each other in the opening and closing portion are attachably and detachably connected to each other by an attachment/detachment member.

It is preferable that the attachment/detachment member is a hook-and-loop fastener or a slide fastener.

It is preferable that regarding one end and the other end of the sheet facing each other in the opening and closing portion, the one end is disposed on an outer side, the other end is disposed on an inner side, and the one end overlaps the other end to cover and conceal the other end.

It is preferable that the frame members are attachably and detachably connected to each other by fitting one end and the other end of the frame members facing each other in the opening and closing portion.

It is preferable that the cover includes a bending portion that bends in a case where the opening and closing portion is opened.

It is preferable that at the bending portion, the frame member is separated, and only the sheet is continuous.

It is preferable that at the bending portion, the frame member is separated and connected by a flexible connecting member.

It is preferable that the cover has a rectangular shape having a short side and a long side in a top view, and the opening and closing portion is provided on the short side.

It is preferable that the opening and closing portion is provided on the short side on a gantry side out of two short sides.

It is preferable that the cover has a rectangular shape having a short side and a long side in a top view, and the opening and closing portion is provided on the long side.

It is preferable that a plurality of the opening and closing portions are provided.

It is preferable that the pedestal part is a caster portion to which casters are attached.

It is preferable that the couch is attachably and detachably connected to a gantry.

According to the technology of the present disclosure, it is possible to provide a couch of a medical image capturing apparatus in which a cover can be easily attached and detached.

### BRIEF DESCRIPTION OF THE DRAWINGS

Exemplary embodiments according to the technique of the present disclosure will be described in detail based on the following figures, wherein:

FIG. 1 is a perspective view of an MRI apparatus;

FIG. 2 is a side view of the MRI apparatus;

FIG. 3 is a perspective view of a cover in a state in which an opening and closing portion is closed;

FIG. 4 is a perspective view of the cover in a state in which the opening and closing portion is opened;

FIG. 5 is a plan view of the cover on a side where the opening and closing portion is provided;

FIG. 6 is a cross-sectional view of the cover on the side where the opening and closing portion is provided;

FIG. 7 is a view showing how frame members are attachably and detachably connected to each other by fitting one end and the other end of the frame members facing each other in the opening and closing portion;

FIG. 8 is a plan view of the cover on a side where a bending portion is provided;

FIG. 9 is a view showing an aspect in which a slide fastener is used as an attachment/detachment member;

FIG. 10 is a view showing an aspect in which the frame members in the bending portion are connected by a rubber tube;

FIG. 11 is a view showing an aspect in which one end and the other end of the frame members facing each other in the opening and closing portion are attachably and detachably connected to each other by a clip;

FIG. 12 is a view showing an aspect in which one end and the other end of the frame members facing each other in the opening and closing portion are attachably and detachably connected to each other by a key-type engagement portion;

FIG. 13 is a view showing the cover provided with the opening and closing portion on a long side;

FIG. 14 is a view showing the cover provided with the opening and closing portion at a corner;

FIG. 15 is a view showing the cover provided with a plurality of the opening and closing portions; and

FIG. 16 is a view showing the cover having a plurality of the bending portions.

### DETAILED DESCRIPTION

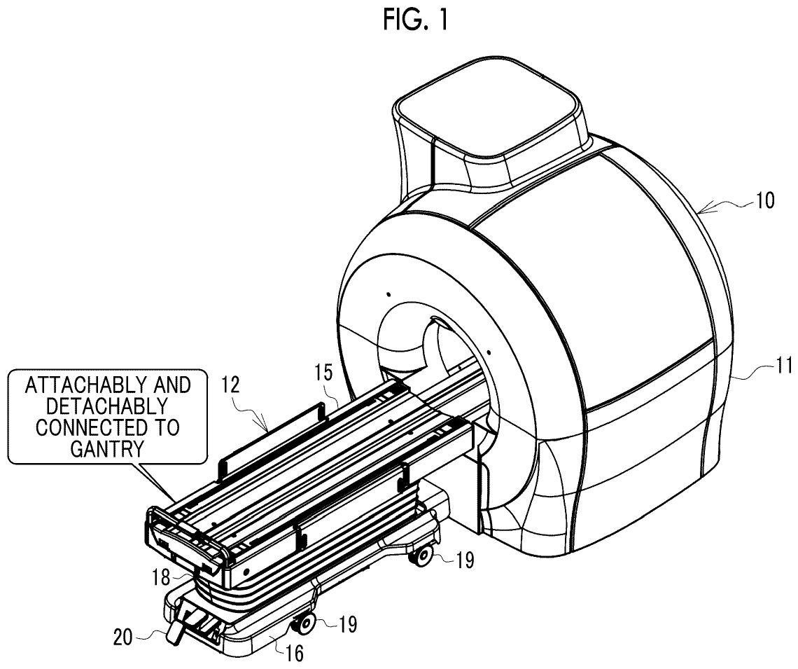

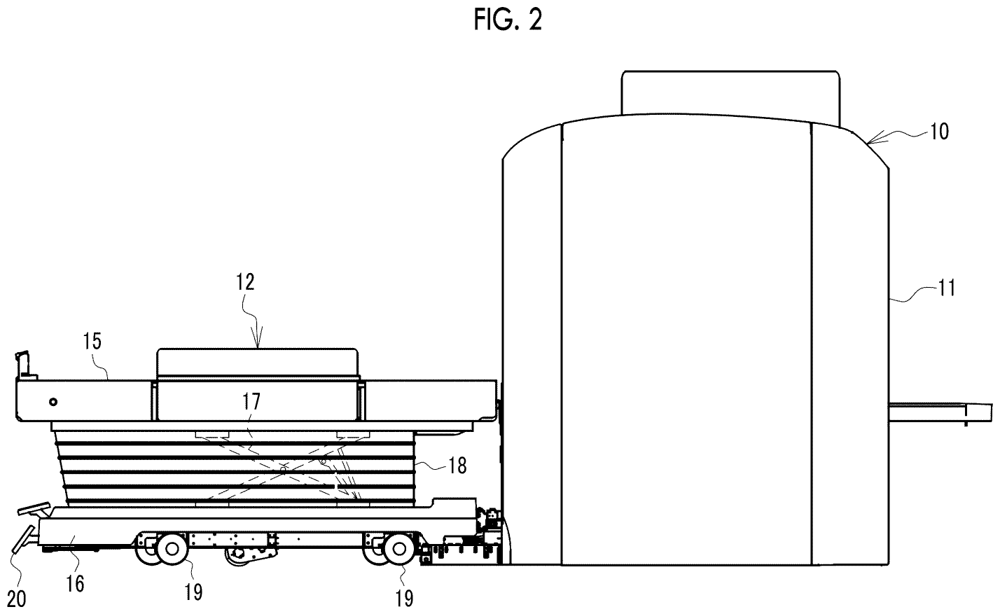

As an example, as shown in FIGS. 1 and 2, an MRI apparatus 10 includes a gantry 11 and a couch 12. The gantry 11 has a cylindrical shape having an imaging space that is a cavity having a circular cross section in a center. The gantry 11 incorporates a magnet and a coil that generate a magnetic field in the imaging space, a detector that detects a nuclear magnetic resonance signal emitted from a subject passing through the imaging space according to the magnetic field, and the like. The MRI apparatus 10 is an example of a “medical image capturing apparatus” according to the technology of the present disclosure. In addition, the couch 12 is an example of a “couch of a medical image capturing apparatus” according to the technology of the present disclosure.

The couch 12 comprises a top plate 15, a caster portion 16, an elevating mechanism 17 (see FIG. 2), a cover 18, and the like. The subject lies on the top plate 15. The caster portion 16 is installed on a floor surface side opposite to the top plate 15. A plurality of casters 19 are attached to the caster portion 16. The couch 12 can travel in a medical facility by the caster portion 16. A foot cover for preventing a foot from hitting the caster 19 is attached to the caster portion 16. The caster portion 16 is an example of a “pedestal part” according to the technology of the present disclosure.

The elevating mechanism 17 is provided between the top plate 15 and the caster portion 16. The elevating mechanism 17 is a so-called scissor lift having an X-shaped pantograph arm connected to the top plate 15 and the caster portion 16 and a cylinder that applies a driving force to the pantograph arm. The elevating mechanism 17 raises and lowers the top plate 15 with respect to the caster portion 16 to adjust a height of the top plate 15. The cover 18 has a substantially quadrangular tubular shape that covers a periphery of the elevating mechanism 17, and can expand and contract vertically according to the driving of the elevating mechanism 17. The couch 12 comprises a slide mechanism for sliding the top plate 15 in the imaging space of the gantry 11, and a fitting such as a belt for fixing a position of the subject to the top plate 15, in addition to the top plate 15, the caster portion 16, the elevating mechanism 17, and the cover 18.

A connector (not shown) is provided on a side of the couch 12 facing the gantry 11. The connector is mechanically and electrically connected to a connector (not shown) of the gantry 11. In addition, a docking pedal 20 is provided at an end part of the caster portion 16 on a side opposite to the gantry 11. The couch 12 can be mechanically and electrically connected to the gantry 11 by pushing the connector of the couch 12 into the connector of the gantry 11 in a state in which the docking pedal 20 is stepped on. In addition, the couch 12 can be detached from the gantry 11 by stepping on the docking pedal 20 again to separate the connector of the couch 12 from the connector of the gantry 11. That is, the couch 12 is a so-called dockable couch that is attachably and detachably connected to the gantry 11 without requiring any work such as attaching and detaching cables.

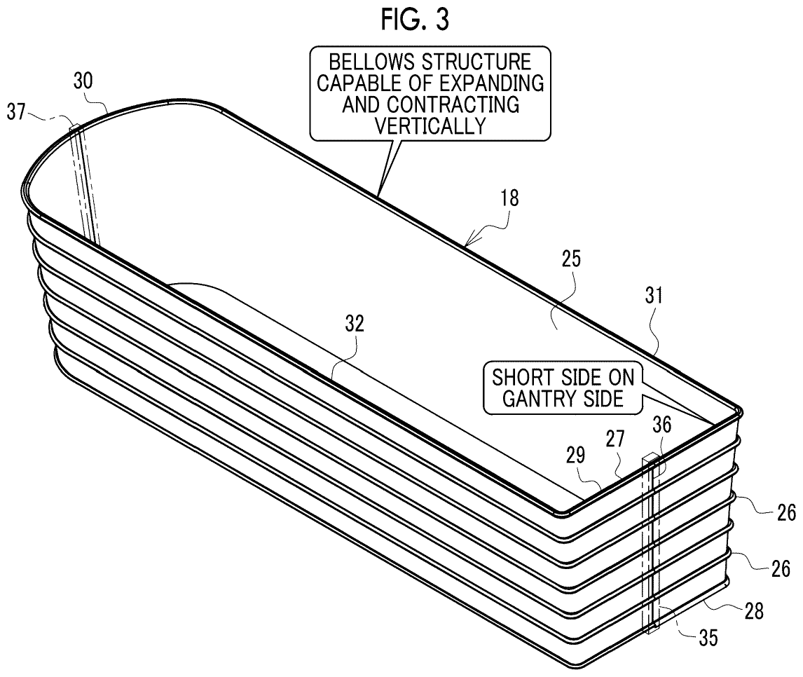

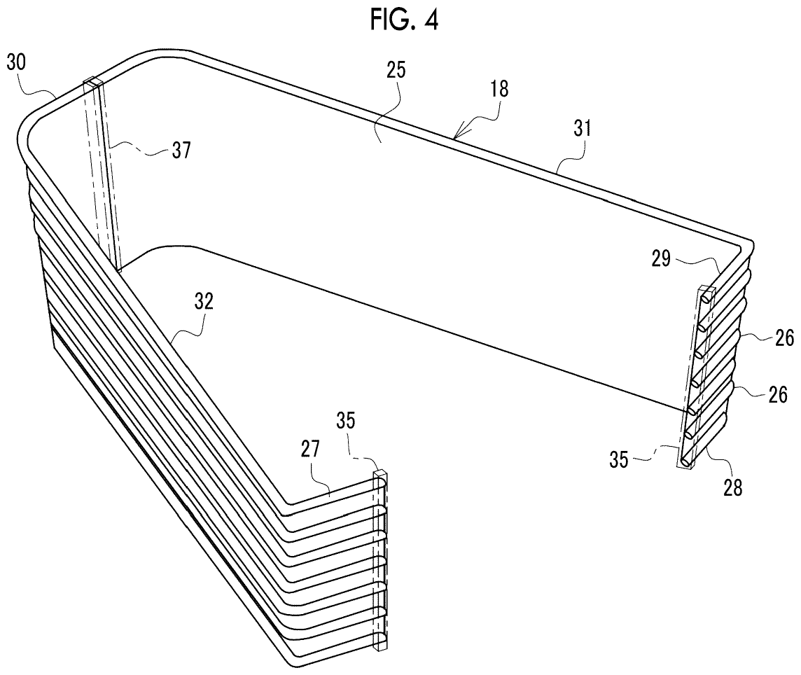

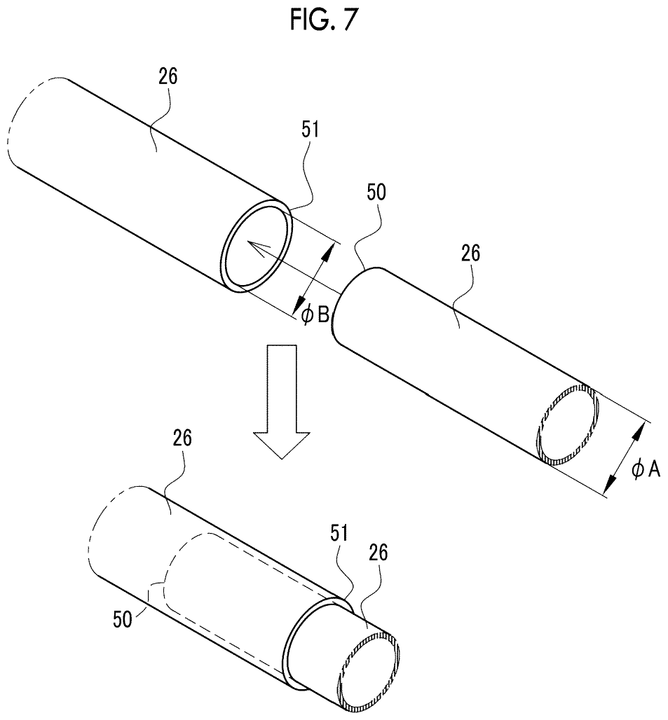

As shown in FIGS. 3 and 4 as an example, the cover 18 has a bellows structure configured with a sheet 25 and a frame member 26. The sheet 25 constitutes the entire cover 18 and is made of a flexible material, for example, cloth, resin, leather, carbon fiber, or the like. A plurality of the frame members 26 are provided at equal intervals from an upper end 27 on the top plate 15 side to a lower end 28 on the caster portion 16 side. The frame member 26 consists of a material having rigidity, for example, an elongated pipe (see FIG. 7) made of metal, contrary to the sheet 25. The frame member 26 extends in a horizontal direction and is bent at places to have a rectangular shape surrounding the periphery of the elevating mechanism 17, and serves as a framework for maintaining a shape of the sheet 25 in a substantially quadrangular tubular shape. In addition, the frame member 26 also serves as a mountain fold portion of the bellows structure in a case where the sheet 25 expands and contracts vertically according to the driving of the elevating mechanism 17. The top plate 15 and the upper end 27, and the caster portion 16 and the lower end 28 are respectively attachably and detachably connected to each other by screws (not shown) at a plurality of locations.

The cover 18 has a rectangular shape having two short sides 29 and 30 and two long sides 31 and 32 in a top view by the frame member 26. The short side 29 of the two short sides 29 and 30 is a short side on the gantry 11 side, and has a straight line shape intersecting the two long sides 31 and 32 having straight line shapes at a right angle. Meanwhile, the short side 30 opposite to the short side 29 has a curved shape having no corners between the two long sides 31 and 32. As described above, the “rectangular shape” according to the technology of the present disclosure also includes a substantially rectangular shape (semi-elliptical shape or elliptical shape) in which a long side and a short side are connected in a curved shape and a boundary between the long side and the short side is not clear. The terms “right angle” and “straight line” refer to a right angle and a straight line in the sense of including an error generally allowed in the technical field to which the technology of the present disclosure belongs, that is, an error to the extent that it does not contradict the gist of the technology of the present disclosure, in addition to a perfect right angle and a straight line.

An opening and closing portion 35 is provided at a center of the short side 29 on the gantry 11 side. The opening and closing portion 35 is formed by a cutout 36 that longitudinally extends through the sheet 25 and the frame member 26 in a vertical line from the upper end 27 to the lower end 28. Therefore, the opening and closing portion 35 can be opened and closed. In addition, a bending portion 37 is provided at a center of the short side 30 facing the opening and closing portion 35. As shown in FIG. 4, the bending portion 37 bends in a case where the opening and closing portion 35 is opened. The opening and closing portion 35 and the bending portion 37 cause the cover 18 to have a structure similar to a blade portion of a nail clipper.

As described above, the frame member 26 extends in the horizontal direction. In addition, the cutout 36 constituting the opening and closing portion 35 longitudinally extends in a straight line shape from the upper end 27 to the lower end 28. Therefore, it can be said that the frame member 26 extends in a direction intersecting the opening and closing portion 35, more specifically, in a direction perpendicular to the opening and closing portion 35. The horizontal direction is an example of a “direction intersecting the opening and closing portion” according to the technology of the present disclosure.

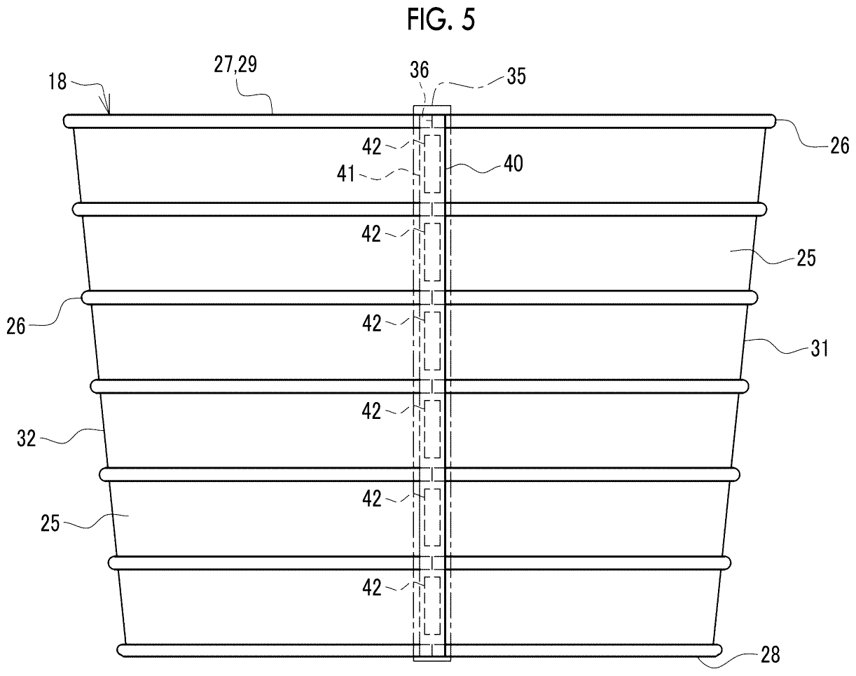

The plurality of frame members 26 have similar shapes that decrease in size from the upper end 27 toward the lower end 28. A plurality of the frame members 26 are arranged such that the centers of the short sides 29 are vertically aligned in an up-down direction. Therefore, in a case where the cover 18 is viewed in a left-right direction, a surface on the short side 30 side is inclined inward from the upper end 27 toward the lower end 28 (see FIG. 2). In addition, in a case where the cover 18 is viewed in a front-rear direction, surfaces on the long sides 31 and 32 sides are inclined inward from the upper end 27 toward the lower end 28 (see FIG. 5 and the like).

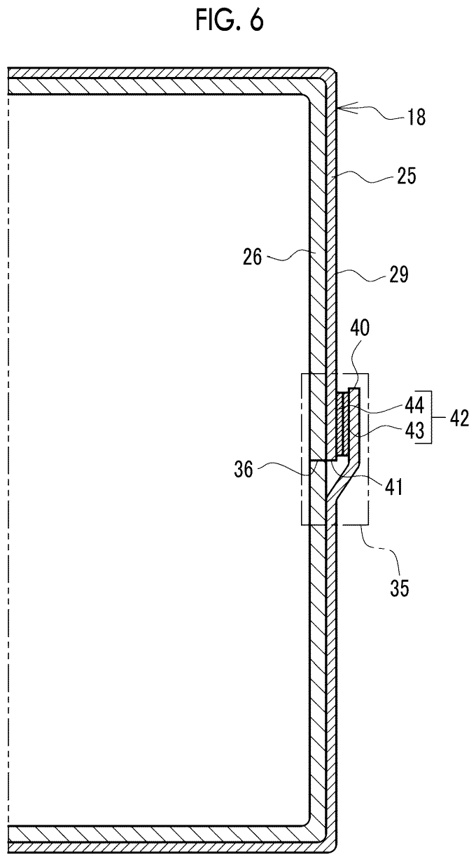

As shown in FIGS. 5 and 6 as an example, one end 40 and the other end 41 of the sheet 25 facing each other in the opening and closing portion 35 are attachably and detachably connected to each other by a hook-and-loop fastener 42. The hook-and-loop fastener 42 is attached between the plurality of frame members 26. The hook-and-loop fastener 42 is configured with a set of a hook 43 attached to the one end 40 and a loop 44 attached to the other end 41 at a position corresponding to the hook 43 (see FIG. 6). The hook-and-loop fastener 42 is an example of an “attachment/detachment member” according to the technology of the present disclosure.

In the opening and closing portion 35, the one end 40 of the sheet 25 is disposed on an outer side and the other end 41 of the sheet 25 is disposed on an inner side, and the one end 40 overlaps the other end 41. Accordingly, the one end 40 covers and conceals the other end 41 and further the hook-and-loop fastener 42 such that they cannot be seen from the outside.

As shown in FIG. 7 as an example, regarding one end 50 and the other end 51 the frame members 26 facing each other in the opening and closing portion 35, an outer diameter φA of the one end 50 is slightly smaller than an inner diameter φB of the other end 51. Therefore, as shown on a lower side of an arrow, the one end 50 can be inserted into the other end 51. That is, the frame members 26 are attachably and detachably connected to each other by fitting the one end 50 and the other end 51 facing each other in the opening and closing portion 35.

In addition, the following configuration may be adopted. That is, inner diameters of the one end 50 and the other end 51 are made to be the same. In addition, a columnar metal piece having an outer diameter slightly smaller than the inner diameter of the one end 50 or the other end 51 is inserted into one of the one end 50 and the other end 51 and is fixed by crimping. Then, the metal piece is inserted into the other of the one end 50 and the other end 51 to fit the one end 50 and the other end 51 to each other. Alternatively, the outer diameters of the one end 50 and the other end 51 are made to be the same. In addition, a cylindrical metal piece having an inner diameter slightly larger than the outer diameter of the one end 50 or the other end 51 is inserted into one of the one end 50 and the other end 51 and is fixed by crimping. Then, the other of the one end 50 and the other end 51 is inserted into the metal piece to fit the one end 50 and the other end 51 to each other.

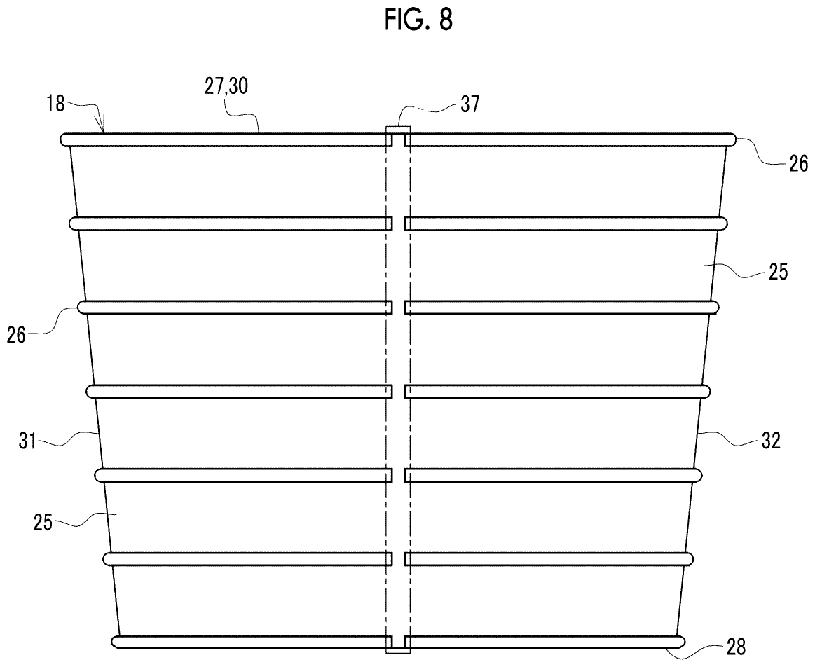

As shown in FIG. 8 as an example, at the bending portion 37, the frame member 26 is separated and only the sheet 25 is continuous. Accordingly, the bending portion 37 is bent in a case where the opening and closing portion 35 is opened.

Next, an operation by the above configuration will be described. First, in a case where the cover 18 is attached to the couch 12, the hook 43 of the hook-and-loop fastener 42 of the one end 40 of the sheet 25 is detached from the loop 44 of the hook-and-loop fastener 42 of the other end 41 of the sheet 25 to release the connection between the one end 40 and the other end 41. In addition, the one end 50 is pulled out from the other end 51 of the frame member 26 to release the fitting between the one end 50 and the other end 51. In this manner, the opening and closing portion 35 can be opened.

Subsequently, as shown in FIG. 4, the opening and closing portion 35 is opened with the bending portion 37 as a fulcrum. Then, the cover 18 is inserted between the top plate 15 and the caster portion 16 from a side opposite to the gantry 11, and the cover 18 is moved in the horizontal direction toward the gantry 11 to cover the elevating mechanism 17 with the cover 18.

Thereafter, the one end 50 is inserted into the other end 51 of the frame member 26, and the one end 50 and the other end 51 are fitted to each other to be connected. In addition, the hook 43 of the hook-and-loop fastener 42 of the one end 40 of the sheet 25 is attached to the loop 44 of the hook-and-loop fastener 42 of the other end 41 of the sheet 25, and the one end 40 and the other end 41 are connected to each other. Further, the top plate 15 and the upper end 27 are connected at a plurality of locations, and the caster portion 16 and the lower end 28 are connected at a plurality of locations, by being fastened and fixed with screws.

The procedure is generally the same in a case of detaching the cover 18 from the couch 12. That is, the hook 43 of the hook-and-loop fastener 42 of the one end 40 of the sheet 25 is detached from the loop 44 of the hook-and-loop fastener 42 of the other end 41 of the sheet 25 to release the connection between the one end 40 and the other end 41. In addition, the one end 50 is pulled out from the other end 51 of the frame member 26 to release the fitting between the one end 50 and the other end 51. Subsequently, the opening and closing portion 35 is opened with the bending portion 37 as a fulcrum. Then, the cover 18 is moved in the horizontal direction toward a side opposite to the gantry 11 to remove the cover 18 from between the top plate 15 and the caster portion 16.

As described above, the couch 12 comprises the top plate 15 on which the subject lies, the caster portion 16 that is installed on the floor surface side, the elevating mechanism 17 that is provided between the top plate 15 and the caster portion 16 and is used to adjust the height of the top plate 15, and the cover 18 that has a tubular shape covering the periphery of the elevating mechanism 17 and is capable of expanding and contracting vertically according to the driving of the elevating mechanism 17. As shown in FIGS. 3 and 4, the cover 18 has the opening and closing portion 35 that is openable and closable and is formed of the cutout 36 longitudinally extending from the upper end 27 on the top plate 15 side to the lower end 28 on the caster portion 16 side.

In the related art, in a case of detaching the cover 18 from the couch 12, in order to fold the cover 18 and slide the cover 18 under the caster portion 16 to be removed, a lot of complicated work such as jacking up the caster portion 16 after detaching the couch 12 from the gantry 11, and inserting a block under the caster portion 16 to ensure a space under the caster portion 16 for the cover 18 to pass through is required. On the other hand, according to the technology of the present disclosure, it is sufficient to open the opening and closing portion 35 and move the cover 18 in the horizontal direction to remove the cover 18. The same applies to a case of attaching the cover 18 to the couch 12. Therefore, the cover 18 can be easily attached and detached. This effect is particularly valuable for the couch 12 of the MRI apparatus 10, which is relatively heavy in weight and places a heavy burden on a worker in handling such as jack-up.

In addition, in the method of the related art, the top plate 15 is attached after the cover 18 is installed during assembly in a factory. Therefore, an assembly worker had to be careful not to cause stains and/or damage to the cover 18 in the work after the cover 18 was installed. On the other hand, according to the technology of the present disclosure, since the cover 18 can be attached after all other work has been completed, the assembly worker can work without hesitation.

In addition, in the method of the related art, there are various design constraints in order to facilitate the attachment and detachment of the cover 18. For example, the cover 18 is formed in a cylindrical shape, and care is taken such that a member such as the docking pedal 20 that protrudes outward is not provided as much as possible in the caster portion 16. On the other hand, according to the technology of the present disclosure, the cover 18 or the like can be freely designed without being subjected to such constraints. That is, in a case of being viewed from the left-right direction, the surface on the short side 30 side can be inclined inward from the upper end 27 toward the lower end 28, and in a case of being viewed from the front-rear direction, the surfaces on the long sides 31 and 32 sides can be inclined inward from the upper end 27 toward the lower end 28. By providing the cover 18 with a gradient in this way, the cover 18 and thus the couch 12 can have a clean appearance, and a feeling of pressure on a patient who is the subject can also be reduced. In addition, the docking pedal 20 or the like can be provided on the caster portion 16 without hesitation.

As shown in FIGS. 3 and 4, the cover 18 has a bellows structure configured with the flexible sheet 25 and the plurality of frame members 26 extending in a direction intersecting the cutout 36. Examples of a structure that can expand and contract vertically according to the driving of the elevating mechanism 17 include a telescopic structure in addition to the bellows structure, but the bellows structure is simpler and cheaper than the telescopic structure. Therefore, according to the bellows structure, it is possible to provide a simple and inexpensive couch 12.

As shown in FIGS. 5 and 6, the one end 40 and the other end 41 of the sheet 25 facing each other in the opening and closing portion 35 are attachably and detachably connected to each other by the hook-and-loop fastener 42. Therefore, the sheet 25 does not hang down loosely in the opening and closing portion 35, and the appearance can be improved. In addition, the hook-and-loop fastener 42 is easy to attach and detach. In addition, since the hook-and-loop fastener 42 is as thin as the sheet 25, the hook-and-loop fastener 42 does not hinder the folding of the sheet 25.

As shown in FIGS. 5 and 6, regarding the one end 40 and the other end 41 of the sheet 25 facing each other in the opening and closing portion 35, the one end 40 is disposed on an outer side and the other end 41 is disposed on an inner side, and the one end 40 overlaps the other end 41 to cover and conceal the other end 41. Therefore, the appearance can be improved, and the user does not have to be conscious of the opening and closing portion 35.

As shown in FIG. 7, the frame members 26 are attachably and detachably connected to each other by fitting the one end 50 and the other end 51 facing each other in the opening and closing portion 35. Therefore, the frame member 26 can be easily attached and detached.

As shown in FIGS. 3 and 4, the cover 18 has the bending portion 37 that bends in a case where the opening and closing portion 35 is opened. Therefore, the opening and closing portion 35 can be easily opened.

As shown in FIG. 8, at the bending portion 37, the frame member 26 is separated and only the sheet 25 is continuous. Therefore, the bending portion 37 can be easily and inexpensively produced.

As shown in FIGS. 3 and 4, the cover 18 has a rectangular shape having the short sides 29 and 30 and the long sides 31 and 32 in a top view, and the opening and closing portion 35 is provided on the short side 29. Therefore, an opening width of the opening and closing portion 35 can be made larger, and the cover 18 can be more easily attached and detached. In addition, an aspect in which the opening and closing portion 35 is provided on the short side 29 is suitable for a case where there are spaces on the left and right sides of the couch 12. In addition, in a case where the opening and closing portion 35 is provided on the short side 30 opposite to the gantry 11, the gantry 11 gets in the way in a case where the cover 18 is attached and detached in a state in which the couch 12 is attached to the gantry 11. On the other hand, according to the technology of the present disclosure, as shown in FIGS. 3 and 4, the opening and closing portion 35 is provided on the short side 29 on the gantry 11 side. Therefore, the gantry 11 does not get in the way in a case where the cover 18 is attached and detached, and the cover 18 can be smoothly attached and detached. In addition, since the opening and closing portion 35 is not noticeable because of the shadow of the gantry 11, the user does not have to be conscious of the opening and closing portion 35.

In the present embodiment, the pedestal part is caster portion 16 to which the casters 19 are attached. Therefore, the couch 12 can be freely moved in the medical facility. The pedestal part is not limited to the caster portion 16 to which the casters 19 are attached, and may be an original pedestal part without the casters 19.

As illustrated in FIG. 1, the couch 12 is a so-called dockable couch that is attachably and detachably connected to the gantry 11. Therefore, it is possible to perform an operation of carrying the couch 12 into a hospital room, moving the subject from a bed in the hospital room to the top plate 15 of the couch 12, and carrying the couch 12 with the subject lying on the top plate 15 into an imaging room of the MRI apparatus 10 to perform imaging. The couch is not limited to the illustrated dockable couch, and may be a stationary couch that is connected to the gantry 11 not to be attachable and detachable.

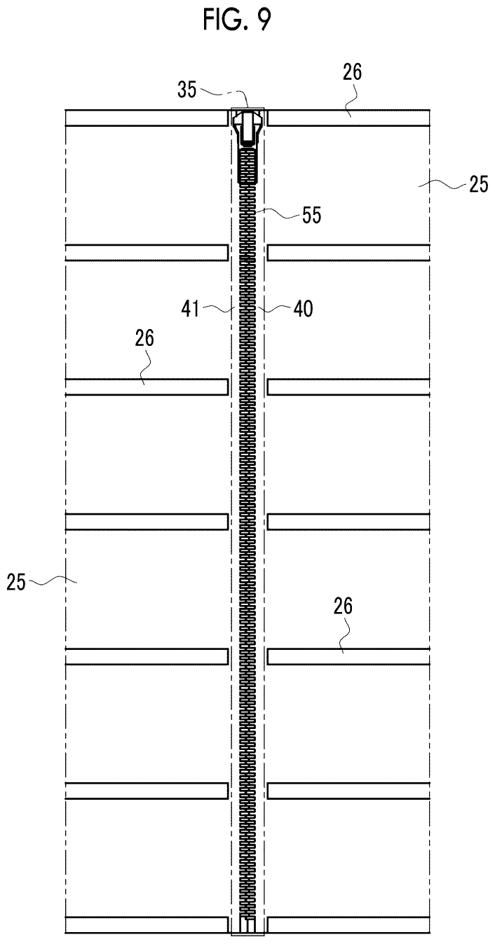

Although the hook-and-loop fastener 42 is illustrated as the attachment/detachment member, the attachment/detachment member is not limited to this. As shown in FIG. 9 as an example, a slide fastener 55 may be adopted instead of the hook-and-loop fastener 42. The slide fastener 55 is easy to attach and detach, similar to the hook-and-loop fastener 42. In addition, in a case of the hook-and-loop fastener 42, there is a concern that the one end 40 and the other end 41 may be displaced in the up-down direction depending on an attachment method, but there is no such concern in a case of the slide fastener 55.

In FIG. 9, the one end 40 is not overlapped with the other end 41, and the other end 41 is not covered and concealed by the one end 40. As described above, it is not essential that the one end 40 overlaps the other end 41 to cover and conceal the other end 41 with the one end 40. Of course, also in FIG. 9, the one end 40 may be overlapped with the other end 41 to cover and conceal the other end 41 with the one end 40.

Similarly, in FIG. 9, the one end 50 and the other end 51 of the frame members 26 are not fitted. As described above, it is not essential that the one end 50 and the other end 51 of the frame members 26 are fitted to each other. At least the one end 40 and the other end 41 of the sheet 25 facing each other in the opening and closing portion 35 need to be attachably and detachably connected to each other by the attachment/detachment member such as the hook-and-loop fastener 42 and the slide fastener 55. Of course, also in FIG. 9, the one end 50 and the other end 51 of the frame members 26 may be fitted to each other.

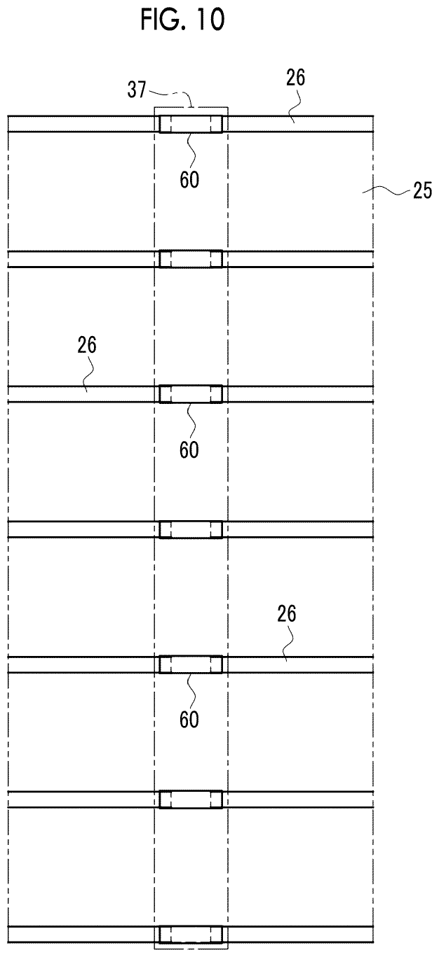

In addition, at the bending portion 37, an aspect in which the frame member 26 is separated and only the sheet 25 is continuous has been illustrated, but the present disclosure is not limited to this. As shown in FIG. 10 as an example, at the bending portion 37, the frame member 26 may be separated and connected by a rubber tube 60. The rubber tube 60 is an example of a “flexible connecting member” according to the technology of the present disclosure. With such a configuration, a strength of the bending portion 37 can be increased as compared with a case where the frame member 26 is separated and only the sheet 25 is continuous.

The frame members 26 are attachably and detachably connected to each other by fitting the one end 50 and the other end 51 facing each other in the opening and closing portion 35, but the present disclosure is not limited to this. As shown in FIG. 11 as an example, the one end 50 and the other end 51 may be attachably and detachably connected to each other by a clip 65.

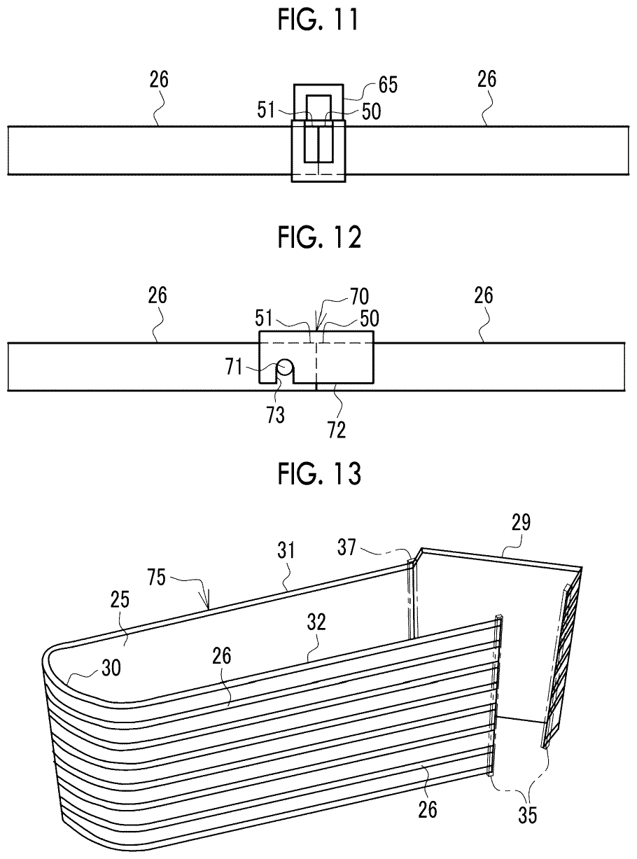

Alternatively, as shown in FIG. 12 as an example, the one end 50 and the other end 51 may be attachably and detachably connected to each other by a key-type engagement portion 70. The key-type engagement portion 70 is configured with a columnar protrusion 71 provided on a peripheral surface of the one end 50 and a rectangular engaging part 72 attached to the other end 51. A notch 73 having a semi-elliptical shape that engages with the protrusion 71 is formed in the engaging part 72. The key-type engagement portion 70 is less likely to come off than an aspect in which the one end 50 and the other end 51 are fitted to each other. In addition, the key-type engagement portion 70 is less bulky than the clip 65.

The short side 29 is illustrated as a location where the opening and closing portion 35 is provided, but the present disclosure is not limited to this. The opening and closing portion 35 may be provided on the short side 30 on the opposite side. In addition, as a cover 75 shown in FIG. 13 as an example, the opening and closing portion 35 may be provided on the long side 32. In this case, the bending portion 37 is provided in a portion of the long side 31 facing the opening and closing portion 35. Here, even in a case where the opening and closing portion 35 and the bending portion 37 are provided on the long sides 32 and 31, it is preferable to provide the opening and closing portion 35 and the bending portion 37 close to the gantry 11 side as shown in FIG. 13 in consideration of case of removal of the cover 18.

In a case where the opening and closing portion 35 is provided on the long side 32, the opening width of the opening and closing portion 35 can be made smaller than in a case where the opening and closing portion 35 is provided on the short side 29 or 30. Even with such a small opening width, the cover 18 can be easily attached and detached. In addition, an aspect in which the opening and closing portion 35 is provided on the long side 32 is suitable for a case where there are spaces on the front and rear sides of the couch 12. The opening and closing portion 35 may be provided on the long side 31, and the bending portion 37 may be provided on the long side 32.

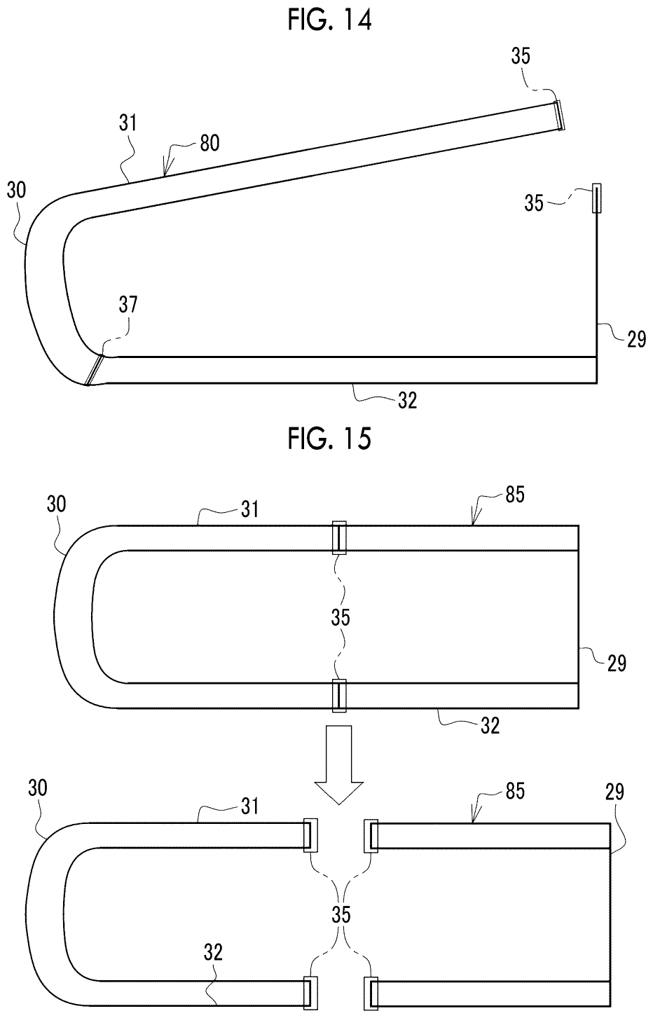

Further, as a cover 80 shown in FIG. 14 as an example, the opening and closing portion 35 may be provided at a corner between the short side 29 and the long side 31. In this case, the bending portion 37 is provided at a corner between the short side 30 and the long side 32, which faces the opening and closing portion 35. With such a configuration, the opening width of the opening and closing portion 35 can be further increased as compared with a case where the opening and closing portion 35 is provided on the short side 29 or 30 and a case where the opening and closing portion 35 is provided on the long side 31 or 32, and the cover 18 can be more easily attached and detached. In addition, an aspect in which the opening and closing portion 35 is provided at the corner is suitable for a case where there are spaces on the left and right sides of the couch 12. Further, an aspect in which the opening and closing portion 35 is provided at the corner is suitable for a case where the elevating mechanism 17 is relatively large and the elevating mechanism 17 cannot be avoided unless the opening width is made large.

In addition, as a cover 85 shown in FIG. 15 as an example, the opening and closing portions 35 may be provided substantially at centers of the long sides 31 and 32, respectively. In other words, the cover 85 that can be divided into two pieces of a front piece and a rear piece may be adopted. In this case, the bending portion 37 is not necessary. Such a configuration allows for easy of portability in a case of transporting a new cover 85 from the factory to the medical facility as a substitute for the stained and/or damaged cover 85. In addition, the cover 85 does not take up much space in a case of being stored in the factory or the medical facility. The opening and closing portion 35 may be provided at a center of the short side 29 and/or 30 instead of or in addition to the centers of the long sides 31 and 32. In addition, a plurality of the opening and closing portions 35 may be provided at any location, not limited to the center of the side.

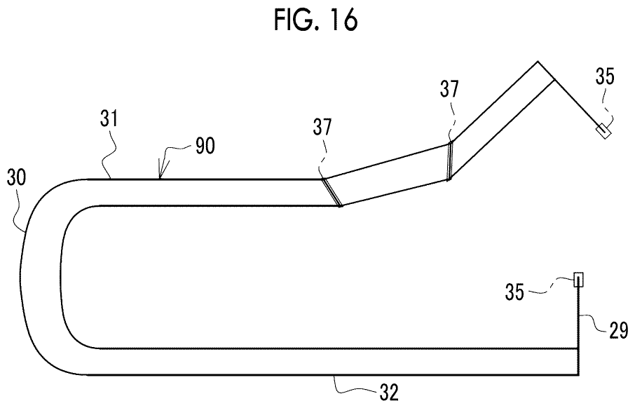

As a cover 90 shown in FIG. 16 as an example, the opening and closing portion 35 is provided on the short side 29, but a plurality of the bending portions 37 may be provided on the long side 31 instead of the short side 30. In short, the bending portion 37 does not need to be provided at a location facing the opening and closing portion 35 as long as the opening and closing portion 35 can be easily opened, and the number of the bending portions 37 is not limited to one.

As the attachment/detachment member, instead of the illustrated hook-and-loop fastener 42 and the slide fastener 55, a button such as a four-hole button, a snap button, or a magnet button, or a hook such as a key hook or a spring hook may be used. The hook-and-loop fastener 42 and the button or the hook, or the button and the hook may be used in combination. However, although the magnet button is easy to attach and detach, there is a concern that the magnet button may be attracted to the gantry 11. Therefore, the magnet button should be carefully considered for adoption in a case where the medical image capturing apparatus is the MRI apparatus 10.

As a method of connecting the one end 50 and the other end 51 of the frame members 26 in the opening and closing portion 35, a method of attaching magnets to the one end 50 and the other end 51 and connecting the one end 50 and the other end 51 by the magnets may be adopted instead of the fitting shown in FIG. 7, the clip 65 shown in FIG. 11, and the key-type engagement portion 70 shown in FIG. 12. However, even in this case, there is a concern that the magnet may be attracted to the gantry 11. Therefore, in a case where the medical image capturing apparatus is the MRI apparatus 10, adoption of this method should be carefully considered.

As a configuration of the bending portion 37, a configuration in which the separated frame members 26 are connected to each other by a hinge may be adopted instead of the configuration in which the frame member 26 is separated and only the sheet 25 is continuous as shown in FIG. 8 and the configuration in which the frame member 26 is separated and connected by the rubber tube 60 as shown in FIG. 10. In a case of the hinge, the strength of the bending portion 37 can be increased as compared with a case of the rubber tube 60. However, the hinge is more complicated in configuration and expensive than the rubber tube 60.

The cutout 36 may be in the form of an oblique straight line or may be in the form of a step. In addition, the cutout 36 may be in the form of a square wave or a sawtooth wave with periodic unevenness.

The cover may be configured with only the sheet 25 without the frame member 26. That is, the cover may not have a bellows structure.

Although the MRI apparatus 10 has been illustrated as the medical image capturing apparatus, the present disclosure is not limited to this. A CT apparatus, a positron emission tomography (PET) apparatus, a single photon emission computed tomography (SPECT) apparatus, or the like may be used.

From the above description, techniques according to the following additional notes can be understood.

### Additional Note 1

A couch of a medical image capturing apparatus, comprising: a top plate on which a subject lies; a pedestal part that is provided on a floor surface side; an elevating mechanism that is provided between the top plate and the pedestal part and is used to adjust a height of the top plate; and a cover that is tubular to cover a periphery of the elevating mechanism, is capable of expanding and contracting vertically according to driving of the elevating mechanism, and has an opening and closing portion that is openable and closable portion and is configured by a cutout longitudinally extending from an upper end on a top plate side to a lower end on a pedestal part side.

### Additional Note 2

The couch of a medical image capturing apparatus according to Additional note 1, in which the cover has a bellows structure configured by a flexible sheet and a plurality of frame members extending in a direction intersecting the opening and closing portion.

### Additional Note 3

The couch of a medical image capturing apparatus according to Additional note 2, in which one end and the other end of the sheet facing each other in the opening and closing portion are attachably and detachably connected to each other by an attachment/detachment member.

### Additional Note 4

The couch of a medical image capturing apparatus according to Additional note 3, in which the attachment/detachment member is a hook-and-loop fastener or a slide fastener.

### Additional Note 5

The couch of a medical image capturing apparatus according to any one of Additional notes 2 to 4, in which regarding one end and the other end of the sheet facing each other in the opening and closing portion, the one end is disposed on an outer side and the other end is disposed on an inner side, and the one end overlaps the other end to cover and conceal the other end.

### Additional Note 6

The couch of a medical image capturing apparatus according to any one of Additional notes 2 to 5, in which the frame members are attachably and detachably connected to each other by fitting one end and the other end of the frame members facing each other in the opening and closing portion.

### Additional Note 7

The couch of a medical image capturing apparatus according to any one of Additional notes 2 to 6, in which the cover has a bending portion that bends in a case where the opening and closing portion is opened.

### Additional Note 8

The couch of a medical image capturing apparatus according to Additional note 7, in which at the bending portion, the frame member is separated, and only the sheet is continuous.

### Additional Note 9

The couch of a medical image capturing apparatus according to Additional note 7, in which at the bending portion, the frame member is separated and connected by a flexible connecting member.

### Additional Note 10

The couch of a medical image capturing apparatus according to any one of Additional notes 1 to 9, in which the cover has a rectangular shape having a short side and a long side in a top view, and the opening and closing portion is provided on the short side.

### Additional Note 11

The couch of a medical image capturing apparatus according to Additional note 10, in which the opening and closing portion is provided on the short side on a gantry side out of two short sides.

### Additional Note 12

The couch of a medical image capturing apparatus according to any one of Additional notes 1 to 11, in which the cover has a rectangular shape having a short side and a long side in a top view, and the opening and closing portion is provided on the long side.

### Additional Note 13

The couch of a medical image capturing apparatus according to any one of Additional notes 1 to 12, in which a plurality of the opening and closing portions are provided.

### Additional Note 14

The couch of a medical image capturing apparatus according to any one of Additional notes 1 to 13, in which the pedestal part is a caster portion to which casters are attached.

### Additional Note 15

The couch of a medical image capturing apparatus according to any one of Additional notes 1 to 14, in which the couch is attachably and detachably connected to a gantry.

The above-described various embodiments and/or various modification examples can be combined as appropriate in the technology of the present disclosure. In addition, it is a matter of course that the technology of the present disclosure is not limited to the above-described embodiment and various configurations can be adopted without departing from the gist.

The described contents and shown contents above are the detailed description of portions according to the technology of the present disclosure, and are merely an example of the technology of the present disclosure. For example, the above descriptions relating to configuration, function, operation, and advantageous effects are descriptions relating to examples of configuration, function, operation, and advantageous effects of the portions according to the technique of the present disclosure. Thus, it goes without saying that unnecessary portions may be removed, or new elements may be added or replaced in the above described contents and the illustrated contents without departing from the gist of the technology of the present disclosure. In order to avoid complications and facilitate understanding of the portion according to the technology of the present disclosure, the description related to common technical knowledge not requiring special description in order to implement the technology of the present disclosure is omitted in the above described content and illustrated content.

In the present specification, “A and/or B” has the same meaning as “at least one of A or B”. That is, “A and/or B” may mean only A, may mean only B, or may mean a combination of A and B. In addition, in the present specification, the same approach as “A and/or B” is applied to a case where three or more matters are represented by connecting the matters with “and/or”.

All documents, patent applications, and technical standards described in the present specification are incorporated in the present specification by reference to the same extent as in a case where each of the documents, patent applications, and technical standards are specifically and individually indicated to be incorporated by reference.

What is claimed is:

1. A couch of a medical image capturing apparatus, comprising:
a top plate on which a subject lies;
a pedestal part that is provided on a floor surface side;
an elevating mechanism that is provided between the top plate and the pedestal part and is used to adjust a height of the top plate; and
a cover that is tubular to cover a periphery of the elevating mechanism, is capable of expanding and contracting vertically according to driving of the elevating mechanism, and has an opening and closing portion that is openable and closable portion and is configured by a cutout longitudinally extending from an upper end on a top plate side to a lower end on a pedestal part side.

2. The couch of a medical image capturing apparatus according to claim 1,
wherein the cover has a bellows structure configured by a flexible sheet and a plurality of frame members extending in a direction intersecting the opening and closing portion.

3. The couch of a medical image capturing apparatus according to claim 2,
wherein one end and the other end of the sheet facing each other in the opening and closing portion are attachably and detachably connected to each other by an attachment/detachment member.

4. The couch of a medical image capturing apparatus according to claim 3,
wherein the attachment/detachment member is a hook-and-loop fastener or a slide fastener.

5. The couch of a medical image capturing apparatus according to claim 2,
wherein regarding one end and the other end of the sheet facing each other in the opening and closing portion, the one end is disposed on an outer side, and the other end is disposed on an inner side, and
the one end overlaps the other end to cover and conceal the other end.

6. The couch of a medical image capturing apparatus according to claim 2,
wherein the frame members are attachably and detachably connected to each other by fitting one end and the other end of the frame members facing each other in the opening and closing portion.

7. The couch of a medical image capturing apparatus according to claim 2,
wherein the cover has a bending portion that bends in a case where the opening and closing portion is opened.

8. The couch of a medical image capturing apparatus according to claim 7,
wherein at the bending portion, the frame member is separated, and only the sheet is continuous.

9. The couch of a medical image capturing apparatus according to claim 7,
wherein at the bending portion, the frame member is separated and connected by a flexible connecting member.

10. The couch of a medical image capturing apparatus according to claim 1,
wherein the cover has a rectangular shape having a short side and a long side in a top view, and
the opening and closing portion is provided on the short side.

11. The couch of a medical image capturing apparatus according to claim 10,
wherein the opening and closing portion is provided on the short side on a gantry side out of two short sides.

12. The couch of a medical image capturing apparatus according to claim 1,
wherein the cover has a rectangular shape having a short side and a long side in a top view, and
the opening and closing portion is provided on the long side.

13. The couch of a medical image capturing apparatus according to claim 1,
wherein a plurality of the opening and closing portions are provided.

14. The couch of a medical image capturing apparatus according to claim 1,
wherein the pedestal part is a caster portion to which casters are attached.

15. The couch of a medical image capturing apparatus according to claim 1,
wherein the couch is attachably and detachably connected to a gantry.
